(19) United States
(12) Patent Application Publication
(10) Pub. No.: US 2014/0255385 A1
(43) Pub. Date: Sep. 11, 2014

(54) TREATMENT OF GLIOBLASTOMA WITH PRODUCTS OBTAINED FROM CULTIVATION OF CORDYCEPS MILITARIS

(71) Applicant: I SHOU UNIVERSITY, Kaohsiung City (TW)

(72) Inventors: Chih-Hui YANG, Kaohsiung City (TW); Keng-Shiang HUANG, Kaohsiung City (TW); Ya-Hui HSIEH, Kaohsiung City (TW)

(73) Assignee: I SHOU UNIVERSITY, Kaohsiung City (TW)

(21) Appl. No.: 13/785,517

(22) Filed: Mar. 5, 2013

(51) Int. Cl. A61K 36/062

(52) U.S. Cl. CPC A61K 36/062
USPC 424/115

(57) ABSTRACT

Disclosed herein is that an active ingredient obtained from cultivation of Cordyceps militaris can be used in the treatment of glioblastoma.

### FIELD OF THE INVENTION

The present invention relates to a treatment of glioblastoma with products obtained from cultivation of cordyceps militaris.

### BACKGROUND OF THE INVENTION

Cancer is one of the major causes of human mortality. Although the formation mechanism of cancer is still not fully understood, the occurrence of cancer may be a result of genetic abnormalities caused by accumulated exogenous or endogenous factors of the cells, and the signal transmission path in the cells has errors, so that cell division loses control, and finally the cells gradually become cancer cells.

Apoptosis is considered as a mechanism for animal cells to control the growth of cells naturally and its main characteristics include cell shrinkage, apoptotic body formation and chromatin condensation, etc. Apoptosis plays an important role in the developmental processes, the maintenance of homeostasis and the elimination of damaged cells of various different cells. Therefore, if the cells have changes and lose the control of the apoptosis mechanism, the cells will be proliferated abnormally to form cancer cells.

In recent years, apoptosis becomes an important research subject of oncology, and researchers all over the world spare no effort on developing anti-cancer drugs for inducing apoptosis of cancer cells. However, the effect of using anti-cancer drugs in Western medicines for cancer treatments is still low, primarily due to the major factors including individual differences of each patient, serious side effects of the anti-cancer drugs and drug resistance of cancer cells. In view of these factors, many researches start attempting to find active components from traditional Chinese medicines (TCM) or herbs to treat cancers.

Entomopathogenic fungi also known as entomogenous fungi are fungi parasitic on insects and with a high host specificity of insects, and these fungi do not infect animals or plants other than insects. The route of infection of entomopathogenic fungi is mainly through an attachment of entomopathogenic fungi in form of microscopic spores onto an external body surface of an insect. Under appropriate temperature and moisture (usually high moisture), the spores on the external body surface of the insect will germinate to form hyphae, and these hyphae penetrate through the cuticle of the insect and enter into the body cavity of the insect (which is the hemocoel). The intruding hyphae grow and multiple by absorbing the nutrients in the insect's body, such that the tissues and organs of the insect are damaged to cause death of the insect.

Entomopathogenic fungi are commonly used as a biological insecticide for agriculture (In addition, some entomopathogenic fungi are found to have great development value for medical purpose, and the common and/or important entomopathogenic fungi include cordyceps sinensis and cordyceps militaris which include ascomycota, pyrenomycetes, hypocreales, clavicipitaceae, and cordyceps.

Cordyceps sinensis is an extensively used traditional Chinese herbal medicine (CHM) parasitic on larvae of lipidoptera and hepialidae insects. Cordyceps sinensis is known to have effects of resisting anti-cardiac arrhythmia, promoting hemangiectasis, and providing antioxidant, anti-tumor and immunoregulation functions.

Cordyceps militaris (also known as silkworm cordyceps militaris) and northern aweto are traditional Chinese herbs with the nutritious and medical values as good as the cordyceps sinensis, and these herbs are parasitic on pupa of lipidopteran insects. In recent years, researches show that extracts of cordyceps militaris have significant anti-cancer or anti-tumor activity. For example, L. Haemi et al. (2006), Biol. Pharm. Bull., 29:670-674 disclosed the effect of a hot water extract of cordyceps militaris fruiting bodies on HL-60 cell strains of human myeloid leukemia. Experiment results show that the hot water extract of the cordyceps militaris fruiting bodies will induce by an apoptosis of HL-60 cells by active caspase-3 to suppress the growth and proliferation of cancer cells.

C. Y. Jin et al. (2008), J. Microbiol. Biotechnol., 18:1997-2003 discovered that an aqueous extract of cordyceps militaris (AECM) in human breast cancer cell strains MDA-MB-231can induce the apoptosis by the caspase-3 and changing the mitochondrial membrane permeability.

E. P. Sang et al. (2009), Food and Chemical Toxicology, 47:1667-1675 discovered that a water extract of cordyceps militaris (WECM) (that uses a signaling cascade of a death receptor-mediated extrinsic pathway and a mitochondrial-mediated intrinsic caspase pathway to induce an apoptosis of human lung cancer cells A549. Particularly, the apoptosis induced by WECM is correlated with the loss of activity of telomerase.

In addition, an active ingredient such as cordycepin and polysaccharide separated from cordyceps militaris are proven to have different biological activities. For example, reports indicate that polysaccharides have anti-cancer, anti-inflammation and immune functions, lower hypoglycemic and hypolipidemic activities, and have the ability to lower cholesterol; and cordycepin has the anti-tumor and anti-fungi activities.

The inventor of the present invention discovered from researches that the liquid culture of the cordyceps militaris and the mycelium product separated from the liquid culture have the capability of inducing the apoptosis of the glioblastoma cells, in addition to the extract of cordyceps militaris, and the cordycepin and polysaccharide separated from the cordyceps militaris having the anti-cancer activity. Therefore, the liquid culture and mycelium product of the cordyceps militaris are expected to have the function of treating glioblastoma.

### SUMMARY OF THE INVENTION

Therefore, it is a primary objective of the present invention to provide a pharmaceutical composition for suppressing the growth of glioblastoma cells, comprising an active ingredient obtained by a cultivation of cordyceps militaris.

The present invention further provides a method of treating an individual having glioblastoma or being suspected to have glioblastoma, and the method obtains an active ingredient for the individual medication by a cultivation of cordyceps militaris.

The aforementioned and other objectives, technical characteristics and advantages of the present invention will become apparent with the detailed description of preferred embodiments accompanied with the illustration of related drawings as follows.

### BRIEF DESCRIPTION OF THE DRAWINGS

FIG. 1 shows the measured cell activity percentages of GBM 8401 cells, U-87MG cells and MCF-7 cells after being processed by liquid cultures of cordyceps militaris in different concentrations, wherein a control represents the case that the cells are not processed by the liquid culture of the cordyceps militaris; and the culture groups 1 to 5 represent the cases that the cells are processed by the liquid culutres of cordyceps militaris with concentrations of 0.5, 1, 1.5, 2 and 2.5 mg/mL respectively, and “i” indicates that p<0.05;

FIG. 2 shows the measured cell activity percentages of GBM 8401 cells, U-87MG cells and MCF-7 cells after being processed by mycelium products of cordyceps militaris of different concentrations, wherein the control represents the case of cells which are not process by the cordyceps militaris mycelium products; and the mycelium product groups 1 to 5 represent the cases that the cells are processed by the mycelium products of cordyceps militaris with concentrations of 0.5, 1, 1.5, 2 and 2.5 mg/mL respectively, and “*” indicates that p<0.05;

FIG. 3 shows the observed cell morphology of GBM 8401 cells and U-87MG cells after being processed by liquid cultures of cordyceps militaris with different concentrations, wherein the control represents the cases that the cells are not processed by the liquid culture of the cordyceps militaris; and the culture groups 1 and 2 represent the cases that the cells are processed by liquid cultures of cordyceps militaris with concentrations of 1 and 2 mg/mL respectively; and the arrowhead indicates that the cell nucleus is stained into blue color by DAPI; and

FIG. 4 shows the observed change of cells modes of GBM 8401 cells and U-87MG cells after being processed by mycelium products of cordyceps militaris with different concentrations, wherein the control represents the cases that the cells are not processed by the mycelium product of the cordyceps militaris; and the culture groups 1 and 2 represent the cases that the cells are processed by mycelium products of cordyceps militaris with concentrations of 1 and 2 mg/mL respectively, and the arrowhead indicates that the cell nucleus is stained into blue color by DAPI.

### DESCRIPTION OF THE PREFERRED EMBODIMENTS

It is noteworthy that any prior art publication cited here shall not constitute an admission as described below. The prior art publication becomes a part of common general knowledge in Taiwan or any other country.

As to the terms used in this specification, “comprising” refers to “including but not limited to” and “comprises” has a corresponding meaning.

Unless defined otherwise, all technical or scientific terms have the meaning understood by those ordinarily skilled in the art. Numerous modifications and variations of the method and material could be made thereto by those skilled in the art without departing from the scope and spirit of the invention set forth in the claims. Of course, the present invention is not limited to the method and material as described above only.

During the process of developing the drug for treating glioblastoma, the inventor of the present invention discovered that the active ingredient obtained by the cultivation of cordyceps militaris has potentials for industrial application. Therefore, the active ingredient obtained from the cultivation of cordyceps militaris in accordance with the present invention can be applied as a medicine for the suppressing the growth of glioblastoma cells.

In this specification, the term “cordyceps militaris” refers to cordyceps militaris strain (such as those purchased in this country or deposited in an organization of a foreign country) easily obtained by those ordinarily skilled in the art or cordyceps militaris strain separated or purified from a natural source by a conventional microbiological separation method, and also includes those called silkworm cordyceps militaris or northern aweto strains.

In a preferred embodiment of the present invention, the cordyceps militaris strain is cordyceps militaris BCRC 32219 (corresponding to ATCC 26848), and this strain is purchased from Biosource Collection and Research Center (BCRC) of Food Industry Research and Development Institute (FIRDI) of Taiwan.

The cultivation of the cordyceps militaris in accordance with the present invention can be performed by introducing a mycelial inoculum of the cordyceps militaris into a solid culture or a liquid culture.

The terms “cultivation” and “culturing” used herein are interchangeable.

Preferably, the cultivation of the cordyceps militaris is performed by introducing the cordyceps militaris to a liquid culture.

In the present invention, if the liquid culture is used for the cultivation of cordyceps militaris, one of the following liquid culture media can be selected for the cultivation, and these liquid culture media include a synthetic medium and a semi-synthetic medium containing a natural material. The formulae and preparation methods of these liquid culture media fall within the scope of professional training and common technical practice of the persons skilled in the art.

In a preferred embodiment of the present invention, the active ingredient obtained from a liquid culture of the cordyceps militaris includes a culture substance formed after the liquid culture takes place. In another preferred embodiment of the present invention, the active ingredient obtained by the liquid culture of a liquid culture substance of cordyceps militaris includes the cordyceps militaris mycelium separated from the culture substance formed after the liquid culture takes place.

In addition, the active ingredient obtained from the cultivation of a solid culture of cordyceps militaris can be obtained by a solid culture medium common to those skilled in the art. For example, the solid culture medium can be an agar medium including a carbon source, a nitrogen source, an inorganic salt or an equivalent substance applicable for the growth of cordyceps militaris. The choice of the carbon sources, nitrogen sources and inorganic salts falls within the scope of professional trainings and common technical practice of the persons skilled in the art.

The culture substance formed after the active ingredient (including the mycelium separated from the culture substance after the liquid culture takes place) obtained from the cultivation of cordyceps militaris in accordance with the present invention is verified by an in vitro anti-cancer cell test that the cancer cells (particularly the glioblastoma cells) have cytotoxicity. In particular, the liquid culture substance of cordyceps militaris and the mycelium separated from the liquid culture substance have the mechanism of inducing an apoptosis of cancer cells and killing cancer cells, so as to achieve the effect of suppressing the growth of tumor/cancer cells.

Therefore, the present invention provides a pharmaceutical composition for suppressing the growth of glioblastoma cells, comprising an active ingredient obtained from a cultivation of cordyceps militaris. In a preferred embodiment of the present invention, the active ingredient includes a culture substance formed after the liquid culture takes place. In another preferred embodiment of the present invention, the active ingredient includes a cordyceps militaris mycelium separated from the culture substance formed after the liquid culture takes place.

The term “pharmaceutical composition” used in the present invention includes at least one kind of active ingredient obtained from the cultivation of cordyceps militaris, one or more medically acceptable carriers, or any other therapeutic agent depending on different situations. The pharmaceutical composition is further combined with other medicines and used together with other active ingredients to improve the treatment effect. The “active ingredient” as described above refers to a non-toxic ingredient with sufficient and effective quantity to achieve the expected effect of suppressing the growth of glioblastoma cells. The so-called “Effective Quantity” depends on general situations such as age and individual, and an applying mode. Therefore, a precise “Effective Quantity” may not be defined accurately. However, an appropriate “Effective Quantity” of any individual can be determined by the experience of those ordinarily skilled in the art.

In a preferred embodiment of the present invention, the active ingredient of the pharmaceutical composition comprises a cordyceps militaris mycelium with a concentration from 0.5 mg/mL to 2.5 mg/mL. In another preferred embodiment of the present invention, the active ingredient comprises a liquid culture of cordyceps militaris with a concentration from 0.5 mg/mL to 2.5 mg/mL.

The pharmaceutical composition according to the present invention can be manufactured into a dosage form of a parenteral, topical or oral medication by the prior art commonly known to those skilled in the art, and the pharmaceutical composition includes but not limited to an injection such as a sterile aqueous solution or a dispersion, a sterile powder, a tablet, a troche, a pill, a capsule and a similar object.

The following parenteral ways in the groups can be selected according to the pharmaceutical composition of the present invention for the medication. These parenteral medications include an intraperitoneal injection, a subcutaneous injection, an intramuscular injection and an intravenous injection.

The term “Parenteral” refers to the medication not going through any nutrition supplying channel, but the medication is supplied through other ways such as a subcutaneous, intramuscular, intraspinal, or intravenous medication.

In a preferred embodiment of the present invention, the pharmaceutical composition is manufactured in a dosage form of medication applicable for intravenous injections.

In another preferred embodiment of the present invention, the pharmaceutical composition is manufactured in a dosage form applicable for an oral medication.

The pharmaceutical composition of the present invention further comprises a carrier extensively used for medicine manufacturing technologies and generally acceptable in the medical field. The carrier includes a phosphate buffer solution, water, an emulsifier such as oil/water, water/oil, and various different kinds of wetting agents. The medically acceptable carrier also includes any composition approved by United States Federal Government Agencies or listed in United States Pharmacopedia for the use in animals including human beings. For example, the medically acceptable carrier includes one or more of the following agents: a solvent, an emulsifier, a suspending agent, a decomposer, a binding agent, an excipient, a stabilizing agent, a chelating agent, a diluent, gelling agent, a preservative, a lubricant, an absorption delaying agent, a liposome and an equivalent substance.

The present invention also provides a method for treating an individual having or suspected to have glioblastoma, and includes an active ingredient obtained by the cultivation of cordyceps militaris and applied for the individual's medication.

According to the present invention, the medication dose and medication frequency of the aforementioned pharmaceutical composition varies with the following factors, including the seriousness of the treated disease, the medication method, and the body weight, age, physical condition and response of the treated individual. In general, the daily medication dose of the pharmaceutical composition of the present invention is usually 500 mg/Kg of body weight to 5000 mg/Kg of body weight, and the medicine is taken by a single dose or divided into a plurality of doses, and the medication can be an oral medication or a parenteral medication.

Preferred Embodiments are described in details as follows. It is noteworthy that these embodiments are provided for the purpose of illustrating the present invention, but not intended for limiting the scope of the present invention.

### Embodiment

General Experiment Material:

1. A liquid culture medium used in the following embodiment comprises the compositions as listed in Table 1 below.

TABLE 1
Formula of Liquid Culture Media

| Composition | Concentration (g/L) |
|---|---|
| Dextrose | 10 |
| Malt extract | 3 |
| Peptone | 5 |
| Yeast extract | 3 |

The rest is deionized water.

2. In the following embodiment, the cordyceps militaris BCRC 32219 (corresponding to ATCC 26848) was purchased from the Biosource Collection and Research Center (BCRC) of the Food Industry Research and Development Institute (FIRDI) with the address (331, Shin Ping Road, Hsinchu, 300 Taiwan).

3. The Source and Cultivation of Cell Strains:

The human glioblastoma multiform (GBM) cell strain GBM 8401 (BCRC 60163), human glioblastoma cell strain U-87MG (BCRC 60360) and human breast cancer cell strain MCF-7 (BCRC 60436) used in the following embodiment were also purchased from the BCRC of the FIRDI.

The GBM 8401 cell was cultivated in a 75-cm² culture flask containing

RPMI 1640 medium (Hyclone, Grand Island, N.Y., USA) [added with 10% fetal bovine serum (FBS), 1% penicillin and 1% streptomycin], and the U-87MG cell and the MCF-7 cell were cultivated in a 75-cm² culture flask containing Dulbecco's modified eagle's medium (DMEM) (Hyclone, Grand Island, N.Y., USA) (added with 10% FBS, 1% penicillin and 1% streptomycin), and these cells are set with the culture conditions at 37° C. and cultivated in an incubator with 5% CO₂. Fresh medium was changed approximately once every 3˜5 days. If the cell density reaches a confluence of approximately 70-80%, the medium is removed, and phosphate buffered saline (PBS) is used to rinse the cells for two times, and then trypsin -EDTA (from Biological Industry) is added to separate the cell from the bottom of the culture flask, and then fresh medium is added, and the activity of trypsin being sucked by a pipette to flush the medium and separate the cells sufficiently, and then the formed cell suspension is distributed into a new culture flask, and the culture condition is set at 37° C. and cultivated in the incubator with 5% CO₂.

General Experiment Method:

1. Statistical Analysis:

In the following embodiment, the experiment of each group is repeated for 3 times, and the experiment data are represented by an average value±SEM. All data is analyzed by the analysis of variance (ANOVA) to evaluate the difference of each group. If the statistical analysis result is p<0.05, then the result has statistical significance.

Embodiment 1. The liquid culture of cordyceps militaris of the present invention and the cell of the mycelium product are used for a cell viability analysis.

Experiment Method:

A. Preparation of Liquid Culture of Cordyceps Militaris and Mycelium Product:

Firstly, the cordyceps militaris BCRC 32219 is inoculated into a liquid culture medium and cultivated in a constant temperature oscillation incubator (20° C., 150 rpm) for 14˜30 days. The formed culture is used as an inoculum of cordyceps militaris.

And then, 3 mL of the inoculum of cordyceps militaris are mixed uniformly with 3 mL of 2% alginate solution, and then the obtained mixture is filled into a syringe (which is operated together with a No. 23 needle), and a syringe pump (produced by KDS 100, KD Scientific, USA) is provided for pushing the syringe, so that the mixture is squeezed from the needle head and added into a culture flask containing 20 mL of 10% CaC1₂ solution, and then the culture flask is set still at 25° C. for 10 minutes to form the inoculum of cordyceps militaris which serves as basic spherical beads. The formed spherical beads are rinsed by sterile water for three times, and then put into a liquid culture medium and cultivated in a constant temperature oscillation incubator (20° C., 150 rpm) for 1˜6 days and then, a portion of the obtained liquid culture is placed at a temperature of -50° C. for freeze drying (FDU-1100, EYELA), and proceeded with a grinding process to obtain a culture powder. In addition, the remaining liquid culture is centrifuged at 25° C. by a 10,000 g for 30 minutes to remove the supernatant liquid , and the formed precipitate is rinsed by sterile water for three times to obtain a mycelium product. the mycelium product and a freeze drying process at −50° C. is taken place, and ground to form a mycelium product powder.

Finally, the foregoing obtained culture powder and mycelium product powder are separately dissolved in DMEM (added with 10% FBS, 1% penicillin and 1% streptomycin) to prepare a culture solution and a mycelium product solution (having the final concentrations of 0.5, 1, 1.5, 2 and 2.5 mg/mL) for future use. In the cell experiment of the following embodiment, the experiment group of the processed culture solution is called a “Culture Group”, and the cell experiment group of the processed the mycelium product solution is called a “Mycelium Product Group”.

B. Cell Activity Analysis of Cordyceps Militaris Liquid Culture and Mycelium Product:

Firstly, the GBM 8401 cell, the U-87MG cell and the MCF-7 cell are divided into six groups which include one control and five experiment groups (which are the culture groups 1, 2, 3, 4 and 5). Each group of cells are added into a 96-well plate (1×10⁴ cell/well) containing 100 μL of cell medium (wherein the medium used for each cell is the third item of the “general experiment material” and as described in the “source and cultivation of cell strain) and cultivated in an incubator (at 37° C. and in 5% CO2) for 16 to 20 hours, and then each group of medium is removed, and 100 μL of a culture solution of the aforementioned item A with a concentration of 0.5, 1, 1.5, 2 and 2.5 mg/mL are added to the culture groups 1, 2, 3, 4 and 5 separately. As to the control, an equal volume of fresh medium is added.

After each group of cells is cultivated in the incubator (at 37° C. and in 5% CO₂) for 24 hours, 3-[4,5-Dimethylthiazol-2-yl]-2,5-diphenyltetrazolium bromide (MTT, 1 mg/mL, 200 μL) is added into the medium and cultivated for 4 hours, and then the liquid in each well is removed, and then 100 μL of DMSO is added and mixed uniformly to form a mixture and measure a light absorption value (0D₅₉₅) by a microplate reader (Thermo Scientific Basic ELISA Type 354) at a wavelength of 595 nm.

To understand the effect of the mycelium product of cordyceps militaris on cancer cells, the GBM 8401 cell, the U-87MG cell and MCF-7 cell are separately divided into one control and five experiment groups (including the mycelium product groups 1, 2, 3, 4 and 5), and experiments of the liquid cultures are performed according to the aforementioned method, except that the mycelium product solution is used instead of the culture solution.

The cell activity percentage (%) can be calculated by substituting the measured light absorption value (OD₅₉₅) into Equation (1):

A=(B/C)×100 Equation (1)

Where, A=cell activity percentage (%)

B=The measured light absorption value (OD₅₉₅) of each group

C=The measured light absorption value (OD₅₉₅) of the control

50% inhibitory concentration (IC₅₀) refers to the concentration capable of suppressing 50% of the cell growth, and can be calculated by the linear portion of the curve (n=3) of the concentration wherein the liquid culture of cordyceps militaris of the present invention and the mycelium product separated from the liquid culture will reduce the light absorption value of the cells up to 50% (compared with that of the control cell).

And then, the first item “Statistical Analysis” of the aforementioned “General Experiment Method” is used for analyzing the experiment data.

Results:

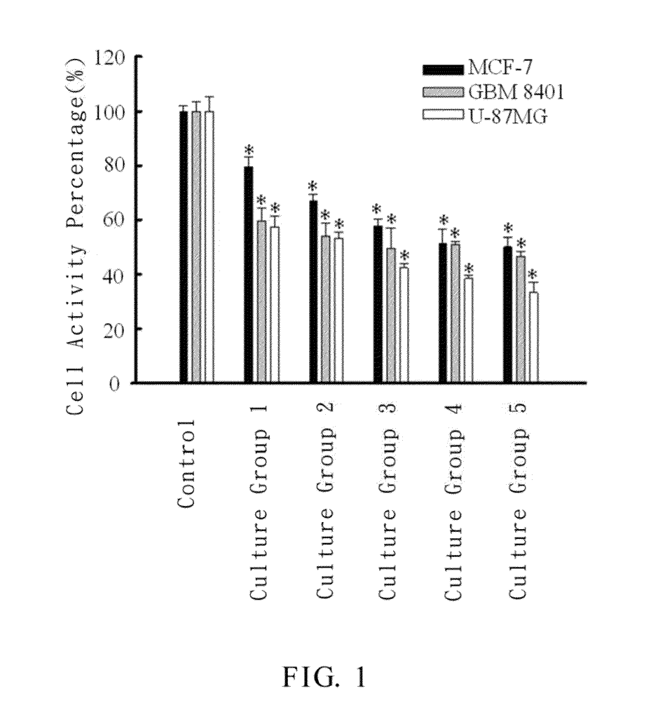

FIG. 1 shows the measured cell activity percentages of GBM 8401 cells, U-87MG cells and MCF-7 cells after being processed by liquid cultures of cordyceps militaris in different concentrations. Compared with the control, the cell activity percentages of the culture groups 1 to 5 drop significantly, and this effect tends to be more significant as the concentration of the liquid culture increases.

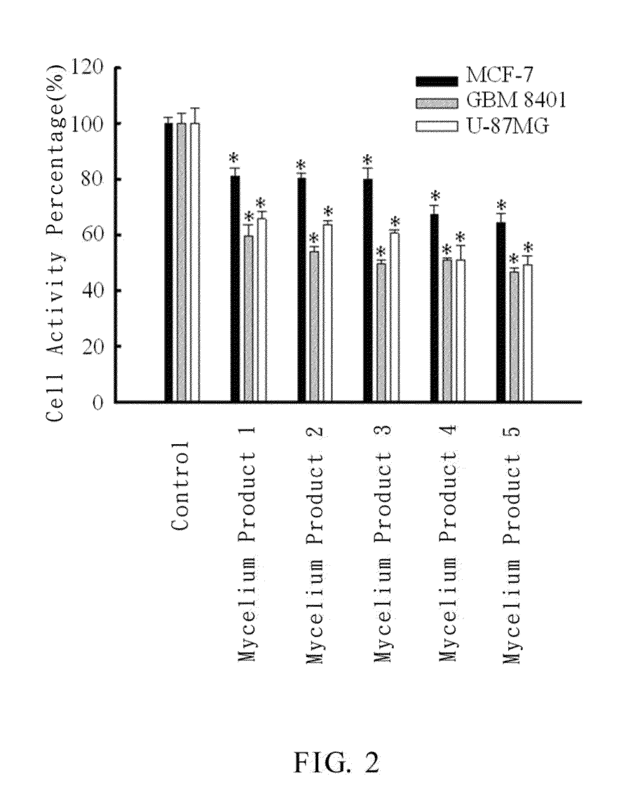

FIG. 2 show the measured cell activity percentages of GBM 8401 cells, U-87MG cells and MCF-7 cells after being processed by mycelium products of cordyceps militaris of different concentrations. Compared with the control, the cell activity percentages of the mycelium product groups 1 to 5 drop significantly, and this effect tends to be more significant as the concentration of the liquid culture increases. The aforementioned experiment results show that the liquid culture of cordyceps militaris and the mycelium product of the present invention are cytotoxic to glioblastoma cells and breast cancer cells. More specifically, the liquid culture of cordyceps militaris and the mycelium product of the present invention is more cytotoxic to glioblastoma cells than breast cancer cells.

The following Table 2 shows the effect of the liquid culture of cordyceps militaris and the mycelium product of the present invention on 50% inhibitory concentration (IC₅₀; mg/mL) of three kinds of cancer cell strains. In Table 2, both liquid culture of cordyceps militaris and mycelium product have the effects of suppressing the growth of tumor/cancer cells and killing tumor/cancer cells. More specifically, the liquid culture of cordyceps militaris has an anti-cancer activity better than that of the mycelium product. Therefore, both liquid culture of cordyceps militaris of the present invention and the mycelium product separated from the liquid culture can suppress the growth and activity of cancer cells for treating cancers, particularly glioblastoma. Based on the experiment results, the inventor of the present invention adopts glioblastoma cells for the following experiments.

TABLE 2
The effect of the liquid culture of cordyceps militaris and the
mycelium product of the present invention on 50% inhibitory
concentration of three kinds of cancer cell strains

| | |
|---|---|
| Cancer cell | IC₅₀ (mg/mL) |

| strain | Liquid culture | Mycelium product |
|---|---|---|
| GBM 8401 | 1.25 ± 0.10 | 1.62 ± 0.09 |
| U-87MG | 1.29 ± 0.08 | 2.28 ± 0.23 |
| MCF-7 | 1.54 ± 0.14 | 2.13 ± 0.04 |

Embodiment 2. The effect of the liquid culture of cordyceps militaris and the mycelium product of the present invention on the apoptosis of human glioblastoma cells is elaborated below.

To evaluate the effect of the liquid culture of cordyceps militaris and the mycelium product of the present invention to induce the apoptosis of human glioblastoma cell strain GBM 8401 and human glioblastoma cell strain U-87MG, the Item A “Preparation of liquid culture of cordyceps militaris and mycelium product” described in the foregoing embodiment 1 is used, and the culture solution and mycelium product solution obtained are used for treating the cells for a flow cytometry.

Experiment Method:

Firstly, the GBM 8401 cells and U-87MG cells are divided into three groups which include one control and two experiment groups (Culture Groups 1 and 2). The cells of each group are added into a 96-well plate (with 1×10⁴ cell/well) containing 100 μL of cell medium (wherein the medium used for each cell is the third item of the “general experiment material” and as described in the third item “source and cultivation of cell strain) and cultivated in an incubator (at 37° C. and in 5% CO₂) for 24 hours, and then each group of medium is removed, and 100 μL of a culture solution of the aforementioned item A with a concentration of 1 mg/mL and 2 mg/mL are added to the culture groups 1 and 2 separately. As to the control, an equal volume of fresh medium is added.

After the cells of each group is cultured in the incubator (37° C., 5% CO₂) for 24 hours, the cell culture is collected and rinsed by PBS twice, and then 70% ethanol below 4° C. is used for fixing the cells for at least two hours, and then all of the fixed cells obtained are rinsed by cold PBS, and then centrifuged at 1500 rpm for 5 minutes. The supernatant liquid is removed and added with 500 μL of cold DNA staining solution [containing 100 μg/mL RNase A solution, 50 μg/mL (propidium iodide, PI), 0.1% (WN) (sodium citrate) and 0.1% (V/V) NP-40] to suspend the cells, and placed on ice in a dark regime for 30 minutes.

The cell cycle analysis of the stained cells is analyzed by a fluorescent activated cell sorting (FACScan, Becton-Dickinson, Mountain View, Calif., USA), wherein a DNA content of 2×10⁶ cells is analyzed each time. Cells are activated by argon-ion laser beams (488 nm) to produce fluorescence. A cell cycle profile obtained from DNA content histograms is analyzed by Cell-Quest™ software (Becton-Dickinson, Mountain View, Calif., USA), wherein GO/G1 peak stands for cells situated at a resting phase of the cell cycle, and they are diploid DNA; G2/M peak stands for cells situated at a mitosis phase, and they are tetraploid DNA; and Sub-G1 peak stands for cells of the apoptosis, and they are hypodiploid DNA.

To evaluate the effect of the mycelium product of cordyceps militaris on inducing the apoptosis of cancer cells, the GBM 8401 cells and U-87MG cells are divided into one control and two experiment groups (including the mycelium product groups 1 and 2), and an experiment of the liquid culture is conducted according to the aforementioned method except that the mycelium product solution is used instead of the culture solution. The method as described in the Item 1 “Statistical Analysis” of the “General Experiment Method” is used for analyzing the obtained experiment data.

Results:

The following Table 3 shows the cell cycle distribution of cells measured after the GBM 8401 cell and the U-87MG cell are treated by the liquid culture or the mycelium product of cordyceps militaris in different concentrations. In Table 3, both liquid culture and mycelium product of the present invention drive the GBM 8401 cells and U-87MG cells to the Sub-G1 phase, and this effect tends to be more significant as the concentration of the liquid culture or the mycelium product increases.

This experiment result shows that the liquid culture of the cordyceps militaris and the mycelium product separated from the liquid culture of the present invention induce the mechanism for the apoptosis of GBM 8401 cell and U-87MG cell and provide a positive dose-effect relation

Therefore, the liquid culture of cordyceps militaris and the mycelium product of the present invention are expected to have the effect of treating glioblastoma.

TABLE 3
Cell cycle distribution measured after GBM 8401 cell and U-87MG
cell are treated by the liquid culture or the mycelium product of the
cordyceps militaris in different concentrations.

| Cancer | | Percentage (%) of Cells Situated at Different | | | |
|---|---|---|---|---|---|
| Cell | | Periodical Stages | | | |
| Strain | Group | Sub-G1 | G0/G1 | S | G2/M |
| GBM | The Control | 6.2 ± 0.3 | 48.0 ± 0.1 | 13.7 ± 0.1 | 32.2 ± 0.2 |
| 8401 | Culture Group | 37.8 ± 0.6 | 36.1 ± 0.8 | 8.9 ± 0.2 | 16.7 ± 0.2 |
| | 1 | | | | |
| | Culture Group | 54.6 ± 1.2 | 26.5 ± 0.8 | 5.6 ± 0.2 | 13.2 ± 0.5 |
| | 2 | | | | |
| | The Control | 6.3 ± 0.8 | 55.9 ± 3.6 | 11.5 ± 0.6 | 26.5 ± 4.9 |
| | Mycelium | 33.1 ± 0.4 | 40.1 ± 1.3 | 9.5 ± 0.4 | 16.8 ± 1.3 |
| | Product Group | | | | |
| | 1 | | | | |
| | Mycelium | 45.3 ± 1.2 | 36.5 ± 2.6 | 8.3 ± 0.7 | 9.8 ± 1.2 |
| | Product Group | | | | |
| | 2 | | | | |
| U- | The Control | 7.4 ± 0.2 | 49.2 ± 0.7 | 16.2 ± 0.2 | 27.2 ± 0.2 |
| 87MG | Culture Group | 10.4 ± 1.9 | 52.3 ± 1.5 | 13.6 ± 1.3 | 23.4 ± 1.2 |
| | 1 | | | | |
| | Culture Group | 56.1 ± 1.4 | 32.3 ± 0.8 | 5.1 ± 0.3 | 6.9 ± 1.2 |
| | 2 | | | | |
| | The Control | 6.9 ± 0.4 | 64.1 ± 3.2 | 11.5 ± 1.2 | 17.4 ± 2.0 |
| | Mycelium | 10.5 ± 3.2 | 58.7 ± 1.2 | 12.8 ± 1.8 | 18.4 ± 1.5 |
| | Product Group | | | | |
| | 1 | | | | |
| | Mycelium | 40.0 ± 0.4 | 32.2 ± 0.5 | 8.9 ± 0.2 | 18.5 ± 0.3 |
| | Product Group | | | | |
| | 2 | | | | |

Embodiment 3. The effect of the liquid culture of cordyceps militaris and the mycelium product of the present invention on the cell morphology of human glioblastoma cells is elaborated below.

To study the cell morphology of the glioblastoma cell strain treated by the liquid culture of cordyceps militaris and the mycelium product of the present invention, the following experiment is conducted.

Experiment Method:

Firstly, the GBM 8401 cells and U-87MG cells are divided into three groups which include one control and two experiment groups (Culture Groups 1 and 2).

The cells of each group are added into a Petri dish (with a diameter of 6 cm and a content of 6×10⁵ cells/dish) containing 2 mL of cell medium (wherein the medium used for each cell is the third item of the “general experiment material” and as described in the third item “source and cultivation of cell strain) and cultivated in an incubator (at 37° C. and in 5% CO₂) for 24 hours, and then each group of medium is removed if the cell density reaches approximately 70˜80% (confluence), and then 2mL of a culture solution of the aforementioned item A in Embodiment 1 with a concentration of 1 mg/mL and 2 mg/mL are added to the culture groups 1 and 2 separately. As to the control, an equal volume of fresh medium is added.

The cells of each group are cultivated in an incubator (37° C., 5% CO₂) for 24 hours, and the cell culture is collected and rinsed by PBS twice, and then 100 μL of 75% iced ethanol is added to fix the cells at room temperature for 2 hours. And then, 100 μL of PBS are used for rinsing the cell culture to remove the iced ethanol. 2.5 mg/mL of 4′,6-diamidino-2-phenylindole (DAPI) solution in PBS is used for nucleus staining and the cell culture is placed in a dark regime at room temperature for 10 minutes, and 100 μL of PBS is used for rinsing the cell culture twice. Finally, a fluorescent microscopy (Nikon, KD 40) with an excitation wavelength 340˜380 nm is used for observing and taking photos of cell morphology.

To evaluate the effect of the mycelium product cordyceps militaris on the cell morphology of cancer cells, GBM 8401 cells and U-87MG cells are divided into one control and two experiment groups (which are the mycelium product groups 1 and 2), and experiments of the liquid culture are conducted according to the aforementioned method except that the mycelium product solution is used instead of the culture solution.

Results:

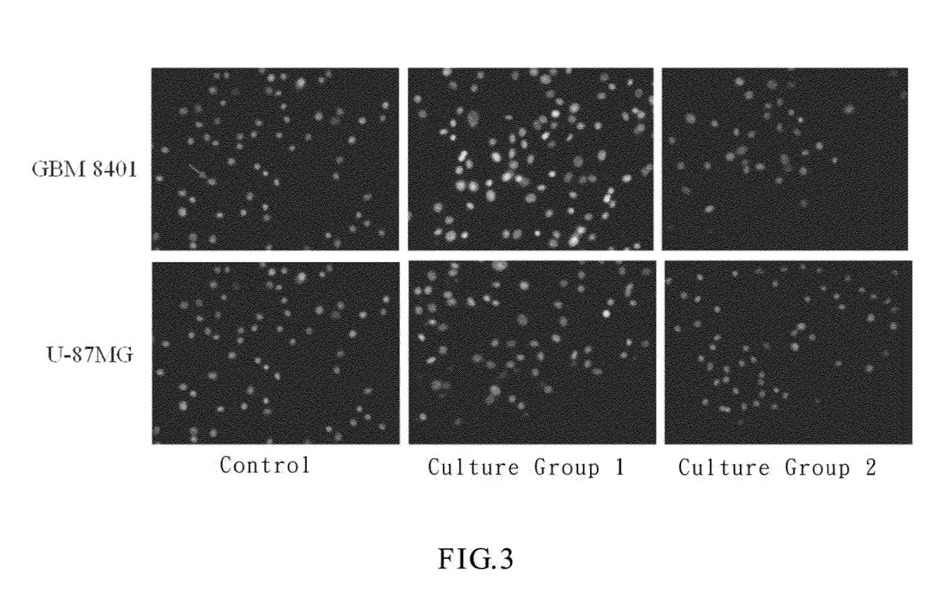

FIG. 3 shows the observed cell morphology of GBM 8401 cells and U-87MG cells after being processed by liquid cultures of cordyceps militaris with different concentrations. Compared with the control, the cells of the culture groups 1 and 2 are observed to have chromatin condensation and apoptotic bodies.

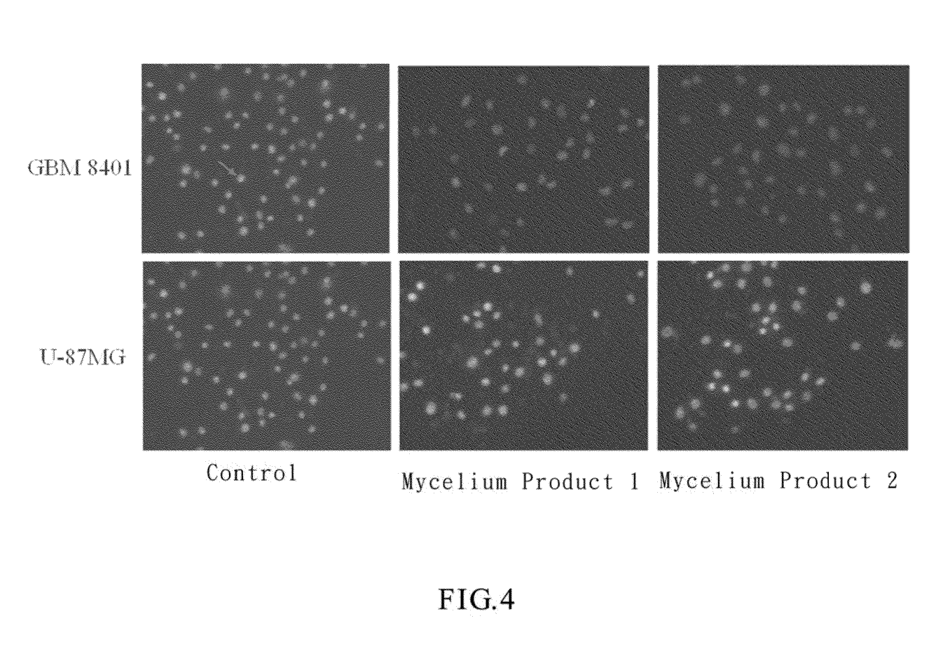

FIG. 4 shows the observed change of cells modes of GBM 8401 cells and U-87MG cells after being processed by mycelium products of cordyceps militaris with different concentrations. Compared with the control, the cells observed in the mycelium product groups 1 and 2 have chromatin condensation and apoptotic bodies.

The foregoing experiment results show that the liquid culture of cordyceps militaris of the present invention and the mycelium product separated from the liquid culture will induce the mechanism for the apoptosis of cancer cells, so as to kill the cancer cells and suppress the growth of cancer cells. Therefore, the liquid culture of cordyceps militaris or the mycelium product of the present invention has a high potential of being developed as an anti-cancer medicine.

All patents and literatures cited in this specification are used as reference of this invention. If there is any conflict, the detailed description of this invention (including definitions) prevails over these references.

While the invention has been described by means of specific embodiments, numerous modifications and variations could be made thereto by those skilled in the art without departing from the scope and spirit of the invention set forth in the claims.

1. A pharmaceutical composition comprising at least one active ingredient and a medically acceptable carrier, wherein the active ingredient is obtained by a cultivation of cordyceps militaris, the active ingredient includes a culture substance with a concentration of 0.5 mg/mL or 1.5˜2.5 mg/mL, and the pharmaceutical composition is used for suppressing the growth of glioblastoma cells.

2. The pharmaceutical composition of claim 1, wherein the cultivation is performed by introducing the cordyceps militaris into a liquid culture.

3. (canceled)

4. (canceled)

5. The pharmaceutical composition of claim 2, wherein the active ingredient includes a cordyceps militaris mycelium separated from the culture substance after the liquid culture takes place.

6. The pharmaceutical composition of claim 5, wherein the active ingredient includes a cordyceps militaris mycelium with a concentration of 0.5 mg/mL or 1.5˜2.5 mg/mL.

7. (canceled)

8. A method for suppressing the growth of glioblastoma cells applied to the pharmaceutical composition as recited in claim 1, and the pharmaceutical composition comprises at least one active ingredient obtained by a cultivation of cordyceps militaris .

9. The method for suppressing the growth of glioblastoma cells according to claim 8, wherein the pharmaceutical composition has a daily dosage from 500 mg/Kg of body weight to 5000 mg/Kg of body weight.

10. The method for suppressing the growth of glioblastoma cells according to claim 8, wherein the cultivation is performed by introducing the cordyceps militaris into a liquid culture.

11. The method for suppressing the growth of glioblastoma cells according to claim 8, wherein the active ingredient includes a culture substance formed after the liquid culture takes place.

12. The method for suppressing the growth of glioblastoma cells according to claim 8, wherein the active ingredient includes a liquid culture substance of cordyceps militaris with a concentration from 0.5 mg/mL to 2.5 mg/mL.

13. The method for suppressing the growth of glioblastoma cells according to claim 8, wherein the active ingredient includes a cordyceps militaris mycelium separated from a culture substance after the liquid culture takes place.

14. The method for suppressing the growth of glioblastoma cells according to claim 8, wherein the active ingredient includes a cordyceps militaris mycelium with a concentration from 0.5 mg/mL to 2.5 mg/mL.

15. The method for suppressing the growth of glioblastoma cells according to claim 8, wherein the pharmaceutical composition is in a dosage form of a parenteral medication or a dosage form of an oral medication.
